(19) United States
(12) Patent Application Publication
(10) Pub. No.: US 2014/0377737 A1
(43) Pub. Date: Dec. 25, 2014

(54) METHOD FOR CULTURING ISLET CELLS AND METHOD FOR PREPARING CARRIER FOR ISLET CELL TRANSPLANTATION USING ATELOCOLLAGEN, AND ARTIFICIAL PANCREAS PREPARED USING SAME

(71) Applicants: Dalim Tissen Inc., Seoul (KR); University of Ulsan Foundation for Industry Cooperation, Ulsan (KR)

(72) Inventors: Song Cheol Kim, Seoul (KR); Si-Nae Park, Seoul (KR); Sun Young Kong, Seoul (KR); Jae-hyung Ko, Seoul (KR)

(21) Appl. No.: 14/381,972

(22) PCT Filed: Mar. 4, 2013
(86) PCT No.: PCT/KR2013/001699

§ 371 Date: Aug. 28, 2014

(30) Foreign Application Priority Data
Mar. 5, 2012 (KR) 10-2012-0022152

(51) Int. Cl. A61K 35/39

(52) U.S. Cl. CPC A61K 35/39
USPC 435/1.1

(57) ABSTRACT

The present invention provides a method of culturing pancreatic islet cells using a cationized atelocollagen prepared by ionization of high purity atelocollagen, a method of preparing a carrier for pancreatic islet cell transplantation using a cationized atelocollagen, and an artificial pancreas prepared using the same. According to the present invention, it is possible to increase the viability and/or glucose-dependent insulin secretion of pancreatic islet cells during culture of the cells by the use of a cationized atelocollagen or crosslinked atelocollagen scaffold obtained by ionization of high purity atelocollagen. In addition, it is possible to increase the viability and glucose-dependent insulin secretion of cultured and transplanted pancreatic islet cells by the use of a highly stable carrier for pancreatic islet cell transplantation that comprises cationized atelocollagen and alginate.

### TECHNICAL FIELD

The present invention relates to a method for culturing pancreatic islet cells and a method for preparing a carrier for pancreatic islet cell transplantation using atelocollagen, and an artificial pancreas prepared using the same, and particularly to a method of culturing pancreatic islet cells using a cationized atelocollagen prepared by ionization of high purity atelocollagen, a method of preparing a carrier for pancreatic islet cell transplantation using a cationized atelocollagen, and an artificial pancreas prepared using the same.

Moreover, the present invention relates to a method of culturing pancreatic islet cells using a cationized atelocollagen or crosslinked atelocollagen scaffold so as to increase the viability and/or glucose-dependent insulin secretion of the pancreatic islet cells, a carrier for pancreatic islet cell transplantation that comprises cationized atelocollagen and alginate, and an artificial pancreas prepared using the same.

In addition, the present invention provides a platform technology for the preparation of an artificial pancreas, which can increase the viability and glucose-dependent insulin secretion of cultured and transplanted pancreatic islet cells by the use of a highly stable carrier for pancreatic islet cell transplantation that comprises cationized atelocollagen and alginate.

### BACKGROUND ART

Patients suffering from diabetes are estimated to account for about 5.1% of global population, and the number of diabetic patients is also expected to increase continuously and account for about 6.3% of global population in 2025. Particularly, it is known that the mortality of diabetic patients reaches 3.1 times that of general people and that diabetes increases the incidence of blindness, chronic renal failure, acute stroke and the like due to its complications, even though it does not lead directly to death. Diabetes is broadly divided into two types: type I diabetes, also called insulin-dependent diabetes, and type II diabetes. Pancreatic islet cells exist in the islet tissue of pancreas, and pancreatic β-cells secrete insulin that plays an essential role in glucose metabolism. It is acknowledged that Type I diabetes is a kind of autoimmune disease that occurs when the immune system destroys β-cells so that insulin required for glucose metabolism is not produced.

Methods for treating type I diabetes, known to date, include a method of injecting insulin at certain intervals of time, and a method of implanting a pancreas from a donor. However, the current cell isolation and culture technology for pancreatic islet cell transplantation remains at a level at which pancreatic islet cells obtained from 2 to 4 donors can be transplanted into one diabetic patient. Also, it is known that, even though transplantation of pancreatic islet cells is successful, the maintenance of insulin independence after the transplantation is less than 10% of people (based on 5 years after transplantation). In order words, insulin that is used for the treatment of type I diabetes has problems that it is expensive and is difficult to be injected by a diabetic patient when required, and causes serious side effects such as shock when it is excessively used. As an alternative thereto, techniques of treating diabetic patients by transplanting pancreatic islets have been developed. However, the supply of the pancreatic islet cells to be transplanted is absolutely insufficient, and for this reason, in the field to which the present invention pertains, studies have been continuously conducted on a method of culturing large amounts of pancreatic islet cells and on a method for preparing artificial pancreatic islets that minimize immune responses.

Meanwhile, it is known that, after transplantation of pancreatic islet cells, a partial or complete loss of the function of the pancreatic islet cells occurs, and the biggest cause of this functional loss of pancreatic islet cells is the destruction of extracellular matrix (ECM) that necessarily occurs when pancreatic islet cells are isolated and purified from the pancreas. Particularly, it is known that extracellular matrix plays an important role not only in the adhesion and migration of cells, but also in signaling for cell stimulation, and for this reason, there are many reports that extracellular matrix greatly increases the adhesion, survival and proliferation of many types of cells, including pancreatic islet cells. Thus, extracellular matrix has received a great deal of attention in the technical field related to the culture and transplantation of pancreatic islet cells and the preparation of artificial pancreases. As a prior art technology that paid attention to this importance of extracellular matrix, Korean Patent Publication No. 10-2003-0033638 discloses a method of preparing artificial pancreatic islet cells by adding pancreatic islet cells to a solution containing a mixture of rat tail collagen and extracellular matrix (ECM) gel. As can be seen from this prior art technology, collagen among extracellular matrix-related biomaterials has been used as an important biomaterial in combination with extracellular matrix. It is known that collagen is distributed in almost all tissues of the body and accounts for about ⅓ of proteins present in the body. Also, it is known that collagen acts as a structure for the support and proliferation of cells and is an essential protein that binds with cells to maintain the form of organs and tissues and to thereby construct the body structure.

Meanwhile, the body has a number of collagen-containing tissues, including skin, ligaments, bone, blood vessels, amnion, pericardium, heart valves, placenta, cornea and the like, but the kind or ratio of collagen slightly differs between tissues. Particularly, type I collagen is abundantly contained in almost all tissues, including skin, ligaments and bone, and thus is an extracellular matrix that has been most widely used in tissue engineering. Further, the inherent properties of collagen can be changed by various chemical treatments. For example, natural collagen does not easily dissolve in neutral water, whereas collagen modified with methanol, ethanol, succinic anhydride, acetic anhydride or the like dissolves even in neutral water since the modified collagen is cationic or anionic. As a technology related to such properties of collagen and the modification of collagen by ionization, U.S. Pat. No. 4,559,304 discloses a technique of ionizing collagen by modifying the amino group and carboxyl group of collagen (for example, preparation of anionic collagen by reacting collagen with succinic anhydride, and preparation of cationized collagen by reacting collagen with alcohol), and discloses that, when mammalian cells are cultured on such ionic collagen, the adhesion and proliferation of the cells are enhanced compared to when native collagen is used. However, U.S. Pat. No. 4,559,304 does not specifically describe a technology related to the culture of pancreatic islet cells, merely mentions the adhesion and proliferation of cells, and neither discloses nor suggests any technical means related to increases in the viability of pancreatic islet cells and the glucose-dependent insulin secretion, which are most important in the culture of pancreatic islet cells.

Generally, it cannot be concluded that, even though cell adhesion and proliferation increase during cell culture, these increases are associated with an increase in cell viability and a positive effect on the function of cells. Particularly, in view of the fact that a pancreatic islet cell is a mass of about 6 types of different cells, which no longer proliferates or differentiates, it cannot be seen that an increase in the adhesion of pancreatic islet cells leads to increases in the viability of pancreatic islet cells and the glucose-dependent insulin secretion (see Example 9 and FIG. 6 in the following description). Thus, in the technical field to which the present invention pertains, there still remains a need for the development of a novel method for culturing pancreatic islet cells to increase the viability of pancreatic islet cells and the glucose-dependent insulin secretion using atelocollagen, and for a highly stable artificial pancreas. Accordingly, the present inventors have conducted studies to develop a technology of increasing the viability and insulin secretory activity of pancreatic islet cells during culture of the cells. As a result, the inventors have found that the viability and glucose-dependent insulin secretory activity of pancreatic islet cells cultured on a cationized atelocollagen scaffold or carrier, which is prepared by cationizing atelocollagen obtained by removing immunogenicity from type I collagen (a representative in vivo extracellular matrix), is much higher than that of pancreatic islet cells cultured on a scaffold and/or carrier prepared from native collagen or anionic collagen to thereby complete the present invention. In addition, the inventors have found that the glucose-dependent insulin secretory activity of pancreatic islet cells cultured on a crosslinked atelocollagen scaffold is higher than that of pancreatic islet cells cultured on a non-crosslinked atelocollagen.

### SUMMARY OF INVENTION

It is an object of the present invention to provide a method of culturing pancreatic islet cells using a cationized atelocollagen prepared by ionizing high purity atelocollagen, a method of preparing a carrier for pancreatic islet cell transplantation using a cationized atelocollagen, and an artificial pancreas prepared using the same.

Still another object of the present invention is to provide a method of culturing pancreatic islet cells using cationized atelocollagen or crosslinked atelocollagen scaffold so as to increase the viability and/or glucose-dependent insulin secretion of the pancreatic islet cells, a carrier for pancreatic islet cell transplantation that comprises cationized atelocollagen and alginate, and an artificial pancreas prepared using the same.

Still another object of the present invention is to provide a platform technology for preparation of an artificial pancreas, which can increase the viability and glucose-dependent insulin secretion of cultured and transplanted pancreatic islet cells by the use of a highly stable carrier for pancreatic islet cell transplantation that comprises cationized atelocollagen and alginate.

### DETAILED DESCRIPTION OF INVENTION

In an embodiment of the present invention, a method for preparing a carrier for pancreatic islet cell transplantation comprises the steps of: (a) mixing a cationized atelocollagen solution with an alginate solution to prepare a mixed solution; (b) adding pancreatic islet cells to the mixed solution of step (a); (c) allowing the pancreatic islet cells to be mixed with and surrounded by the mixed solution of step (a) to form a pancreatic islet cell complex comprising the pancreatic islet cells surrounded by the mixed solution of step (a); and (d) immersing the pancreatic islet cell complex obtained by step (c) comprising the pancreatic islet cells surrounded by the mixed solution of step (a), in a chelating agent solution to chelate the cationized atelocollagen and the alginate in the mixed solution and to produce a cationized atelocollagen/alginate bead containing the pancreatic islet cells therein.

In another embodiment of the present invention, the method for preparing a carrier for pancreatic islet cell transplantation preferably further comprises a step of forming an immune barrier on the cationized atelocollagen/alginate bead of step (d). This immune barrier may serve to prevent or minimize immune responses that are caused by the pancreatic islet cells transplanted into a diabetic patient and to increase the viability of the pancreatic islet cells that are carried by the carrier for pancreatic islet cell transplantation. For example, the immune barrier may be formed by immersing the cationized atelocollagen/alginate bead of step (d) in a poly-L-lysine solution. However, the scope of the present invention is not limited thereto, and any immune barrier may be applied to the cationized atelocollagen/alginate bead, as long as it may be used in cell carriers in the technical field to which the present invention pertains.

In still another embodiment of the present invention, the method for preparing a carrier for pancreatic islet cell transplantation may further comprise a step of forming an additional alginate coating directly on the cationized atelocollagen/alginate bead or the immune barrier. When this additional alginate coating is formed, the cationized atelocollagen/alginate bead has increased its stability as compared with conventional esterified collagen beads, and thus the morphology of the pancreatic islet cells contained therein can be maintained for a long period of time during culture of the cells, whereby the effect of delivering the pancreatic islet cells into a patient can be improved and the viability of the pancreatic islet cells can also be increased.

In one embodiment of the present invention, the chelating agent comprises a metal ion chelating agent that chelates the cationized atelocollagen and the alginate in the mixed solution of the cationized atelocollagen solution and the alginate solution. For example, the chelating agent may be a calcium chloride solution. However, the scope of the present invention is not limited thereto, and any metal ion chelating agent may be used in the present invention, as long as it can chelate cationized atelocollagen and alginate.

In a preferred embodiment of the present invention, the concentration ratio between the cationized atelocollagen solution and the alginate solution, which are mixed in step (a), is preferably 1:2. Meanwhile, in one embodiment of the present invention, the carrier for pancreatic islet cell transplantation may be prepared by, for example, the above-described method for preparing a carrier for pancreatic islet cell transplantation.

In one embodiment of the present invention, the carrier for pancreatic islet cell transplantation preferably further comprises an immune barrier formed on the cationized atelocollagen/alginate bead. More preferably, the carrier for pancreatic islet cell transplantation may further comprise an alginate coating formed directly on the cationized atelocollagen/alginate bead or formed on the immune barrier. In another embodiment of the present invention, an artificial pancreas comprises: a carrier for pancreatic islet cell transplantation in the form of the cationized atelocollagen/alginate bead as described above; and pancreatic islet cells contained in the carrier for pancreatic islet cell transplantation. In another embodiment of the present invention, the artificial pancreas preferably further comprises an immune barrier formed on the cationized atelocollagen/alginate bead of the carrier for pancreatic islet cell transplantation. More preferably, the artificial pancreas may further comprise an alginate coating formed directly on the cationized atelocollagen/alginate bead or formed on the immune barrier.

In one embodiment of the present invention, a method of culturing pancreatic islet cells using atelocollagen comprises the steps of: (a) preparing a cationized atelocollagen solution; (b) either seeding pancreatic islet cells into the cationized atelocollagen solution, or applying the cationized atelocollagen solution to a culture vessel, drying the applied cationized atelocollagen solution to form a cationized atelocollagen scaffold, and seeding pancreatic islet cells onto the cationized atelocollagen scaffold; and (c) culturing the seeded pancreatic islet cells of step (b) in the cationized atelocollagen solution or on the cationized atelocollagen scaffold.

In another embodiment of the present invention, a method of culturing pancreatic islet cells using atelocollagen comprises the steps of: (a) preparing an atelocollagen solution; (b) applying the atelocollagen solution to a culture vessel, drying the applied atelocollagen solution to form an atelocollagen scaffold, crosslinking the atelocollagen scaffold, and seeding pancreatic islet cells onto the crosslinked atelocollagen scaffold; and (c) culturing the seeded pancreatic islet cells of step (b) on the crosslinked atelocollagen scaffold. Preferably, the crosslinking of the atelocollagen scaffold in step (b) may be induced by reacting the atelocollagen scaffold with a solution containing a crosslinking agent. For example, the crosslinking agent may be 1-ethyl-3-(3-dimethyl aminopropyl)carbodiimide (EDC) or glutaraldehyde.

### Advantageous Effects

According to the present invention, pancreatic islet cells can be efficiently cultured using either a cationized atelocollagen obtained by ionization of high purity atelocollagen or a crosslinked atelocollagen scaffold, and a highly stable carrier for pancreatic islet cell transplantation and a highly stable artificial pancreas can be provided using cationized atelocollagen.

According to the present invention, using either a cationized atelocollagen obtained by ionization of high purity atelocollagen or a crosslinked atelocollagen scaffold, the viability and/or glucose-dependent insulin secretion of pancreatic islet cells during culture can be increased, and a highly stable carrier for pancreatic islet cell transplantation that comprises cationized atelocollagen and alginate can be provided, thereby increasing the viability and glucose-dependent insulin secretion of cultured and transplanted pancreatic islet cells.

### BRIEF DESCRIPTION OF DRAWINGS

FIG. 1 shows microscopic images of cultured pancreatic islet cells, obtained with a CKX41 Olympus microscope (Olympus, Tokyo, Japan) at 1 week after culture of pancreatic islet cells on each of a culture dish having a cationized atelocollagen scaffold formed thereon, a culture dish having a native atelocollagen scaffold formed thereon, a culture dish having an anionized atelocollagen scaffold formed thereon, a culture dish having poly-L-lysine formed thereon, and a negative control culture dish.

FIG. 2 shows microscopic images of cultured pancreatic islet cells, obtained with a CKX41 Olympus microscope (Olympus, Tokyo, Japan) at 5 weeks after culture of pancreatic islet cells on each of a culture dish having a cationized atelocollagen scaffold formed thereon, a culture dish having a native atelocollagen scaffold formed thereon, a culture dish having an anionized atelocollagen scaffold formed thereon, a culture dish having poly-L-lysine formed thereon, and a negative control culture dish.

FIG. 3 is a graphic diagram showing a comparison of insulin secretion in terms of insulin concentration measured after low concentration (3.3 mM) and high concentration (20 mM) glucose stimulation of pancreatic islet cells cultured on each of cationized atelocollagen(CC), anionized atelocollagen(AC), native atelocollagen(NC), poly-L-lysine(PLL) and negative control(N). The left graph indicates insulin concentration after low concentration (3.3 mM) glucose stimulation, and the right graph indicates insulin concentration after high concentration (20 mM) glucose stimulation.

FIG. 4 is a graphic diagram showing a comparison of insulin secretion in terms of glucose stimulation index measured after glucose stimulation of pancreatic islet cells cultured on each of cationized atelocollagen(CC), anionized atelocollagen(AC), native atelocollagen(NC), poly-L-lysine(PLL) and negative control(N).

FIG. 5 is a graphic diagram showing a comparison of the number of pancreatic islet cells counted at 1 day, 3 weeks and 8 weeks after culture on each of a cationized atelocollagen scaffold(CC), an anionized atelocollagen scaffold(AC) and a negative control(N) in order to determine the viability of the pancreatic islet cells. The left graph shows the cell number counted at 1 day after culture, the middle graph shows the cell number counted at 3 weeks after culture, and the right graph shows the cell number counted at 8 weeks after culture.

FIG. 6 is a graphic diagram showing the results of MTT assay where the absorbance was measured at 3 days and 7 days after culture of L929 cells, and also showing the results of MTT assay where the absorbance was measured at 3 days and 7 days after culture of rat MSC cells.

FIG. 7 is a graphic diagram showing shows a comparison of insulin secretion in terms of insulin concentration measured at 1 day and 1 week after low concentration (3.3 mM) and high concentration (20 mM) glucose stimulation of pancreatic islet cells cultured on each of a carrier for pancreatic islet cell transplantation of the present invention and an alginate bead as a control. The left graph shows insulation concentration after low concentration (3.3 mM) glucose stimulation, and the right graph shows insulin concentration after high concentration (20 mM) glucose stimulation.

FIG. 8 is a graphic diagram showing a comparison of insulin secretion in terms of glucose stimulation index measured at 1 day and 1 week after glucose stimulation of pancreatic islet cells cultured on each of a carrier for pancreatic islet cell transplantation of the present invention and an alginate bead as a control.

FIG. 9 is a fluorescence microscope image obtained after FDA/PI staining of pancreatic islet cells contained in each of a cationized collagen/alginate bead (that is a carrier for pancreatic islet cell transplantation of the present invention) and an alginate bead as a control.

FIG. 10 is a graphic diagram showing a comparison of insulin secretion in terms of insulation concentration measured after low concentration (3.3 mM) and high concentration (20 mM) glucose stimulation of pancreatic islet cells cultured on each of crosslinked cationized atelocollagen(CLEC), crosslinked anionized atelocollagen(CLSC), crosslinked atelocollagen(native)(CLNC), cationized atelocollagen(EC), native atelocollagen(NC) and negative control(N). The left graph shows insulin concentration after low concentration (3.3 mM) glucose stimulation, and the right graph shows insulin concentration after high concentration (20 mM) glucose stimulation.

FIG. 11 is a graphic diagram showing a comparison of insulin secretion in terms of glucose stimulation index measured after glucose stimulation of pancreatic islet cells cultured on each of crosslinked cationized atelocollagen(CLEC), crosslinked anionized atelocollagen(CLSC), crosslinked atelocollagen(native)(CLNC), cationized atelocollagen(EC), native atelocollagen(NC) and negative control(N).

### EXAMPLES

Hereinafter, the present invention will be described with reference to non-limiting examples. It is to be understood, however, that these examples are for illustrative purposes only and are not intended to limit the scope of the present invention. Thus, those that can be easily contemplated by persons skilled in the art from the detailed description and examples of the present invention are interpreted to fall within the scope of the present invention. References cited herein are incorporated herein by reference.

### Example 1

### Preparation of Cationized Atelocollagen and Anionized Atelocollagen

First, native atelocollagen was prepared by pretreating animal tissue, removing telopeptide from collagen in the pretreated tissue and extracting atelocollagen from the pretreated tissue according to a process well known in the art (see, for example, Korean Patent Publication No. 10-2011-0125772).

### Example 1-1

### Preparation of Cationized Atelocollagen

Cationized atelocollagen used in the culture of pancreatic islet cells and the preparation of a carrier for pancreatic islet cell transplantation in the examples of the present invention was prepared in the following manner.

1) A dispersion of 1-5 wt % of atelocollagen (one isolated and purified according to the method described in Korean Patent Publication No. 10-2011-0125772 or commercially available atelocollagen) in 70-90% ethanol (or methanol) was adjusted to pH 2 to 4 by adding 0.5-1M acetic acid or 0.1-0.5M HCl thereto, and then stirred at 4° C. for 4-10 days.

2) The atelocollagen dispersion obtained in step 1) was adjusted to pH 7.4 with 0.1-0.5M NaOH, and then centrifuged, and the precipitate was collected.

3) The resulting precipitate obtained in step 2) was stirred in purified water in the ratio of 10-100 mL (purified water) per 1 g (precipitate), and then transferred into a dialysis membrane and dialyzed in a dialysis buffer.

4) After stifling for 16-24 hours, the dialysis buffer was replaced, after which the dialysis buffer was replaced 3-12 times at intervals of 3-5 hours each time.

5) The cationized atelocollagen precipitate dialyzed in steps 3) and 4) was freeze-dried at −70° C. for 30 hours or more, and the freeze-dried cationized atelocollagen was collected.

The following reaction scheme 1 shows the reaction in which atelocollagen is cationized by the above-described preparation process:

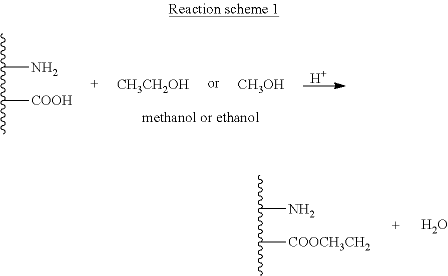

Meanwhile, in a conventional method for preparing cationized atelocollagen, only dialysis with purified water is performed in order to increase the yield and purity of cationized atelocollagen, whereas in a method of preparing cationized atelocollagen according to an embodiment of the present invention, a dispersion of atelocollagen in ethanol or methanol was neutralized and centrifuged, and only the precipitate was collected, and then dialyzed through a dialysis membrane to increase the yield and purity of cationized atelocollagen.

### Example 1-2

### Preparation of Anionized Atelocollagen as Control

Meanwhile, in order to demonstrate the superiority of the method of culturing pancreatic islet cells using cationized atelocollagen and the carrier for pancreatic islet cell transplantation according to the present invention, anionized atelocollagen as a control for comparison was prepared in the following manner.

1) 0.002-0.01 wt % of atelocollagen (one isolated and purified according to the method described in Korean Patent Publication No. 10-2011-0125772 or commercially available atelocollagen) was added to 0.1M acetic acid solution and stirred at 4° C. for 1-2 days to dissolve the atelocollagen.

2) To the atelocollagen solution obtained in step 1), succinic anhydride was added in an amount of 0.8-1.3 g per g of atelocollagen, and the mixture was maintained at about pH 9 to 10 using 0.05-1M NaOH for 10 minutes.

3) The solution obtained in step 2) was stirred at 4° C. for 30 minutes.

4) The solution stirred in step 3) was maintained at about pH 9 to 10 using 0.05-1M NaOH for 10 minutes.

5) The solution obtained in step 4) was stirred at 4° C. for 30 minutes.

6) The solution stirred in step 5) was maintained at about pH 9 to 10 using 0.05-1M NaOH for 10 minutes.

7) The solution obtained in step 6) was stirred at 4° C. for 20 minutes.

8) The solution stirred in step 7) was maintained at about pH 9 to 10 using 0.05-1M NaOH for 10 minutes.

9) The solution obtained in step 8) was stirred at 4° C. for 10 minutes.

10) The solution stirred in step 9) was maintained at about pH 9 to 10 using 0.05-1M NaOH.

11) The solution obtained in step 10) was adjusted to pH 4.03 using 3-7M HCl to form an anionized atelocollagen precipitate, and then stirred at 4° C. for 15 minutes.

12) The solution stirred in step 11) was centrifuged, and the anionized atelocollagen precipitate was collected.

13) To the atelocollagen precipitate obtained in step 12), distilled water adjusted to pH 4.03 with 3-7M HCl was added in an amount of about 20 mL per g of atelocollagen used in step 1), and the mixture was stirred at 4° C. for 15 minutes to wash the atelocollagen precipitate.

14) The solution in step 13) was centrifuged, and the washed anionized atelocollagen was collected.

15) Steps 13) and 14) were repeated once more, and the resulting anionized atelocollagen was freeze-dried at −70° C. for 30 hours, thereby obtaining anionized atelocollagen.

The following reaction scheme 2 shows the reaction in which atelocollagen is anionized by the above-described preparation process:

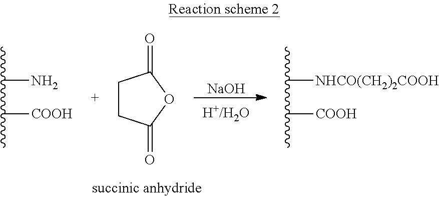

Meanwhile, when anionized collagen is prepared by a conventional method, there is a problem that succinic anhydride is not dissolved at a too low or high pH. Succinic anhydride is most easily dissolved at about pH 9 to 10, but is not dissolved at pH 11 or higher. In view of the problem that the change in pH caused by the reaction between atelocollagen and succinic anhydride leads to a decrease in the solubility of succinic acid, which results in a decrease in reaction rate and yield, the inventors introduced steps 3-11 of maintaining the pH of the reaction solution at 9-10 in a repeated manner.

In other words, in the above-described method for preparing anionized atelocollagen, the reaction solution of atelocollagen and succinic anhydride was stirred at low temperature for a predetermined time, and then the stirred solution was maintained at pH 9 to 10 for a predetermined time, whereby succinic anhydride was easily dissolved to promote the anionization of atelocollagen.

### Example 2

### Culture of Pancreatic Islet Cells on Collagen Scaffold

In order to demonstrate the superiority of the method of culturing pancreatic islet cells using cationized atelocollagen according to the present invention, pancreatic islet cells were cultured on a collagen scaffold in the following manner.

1) 1.5 wt % of type I atelocollagen suspension (i.e. native atelocollagen suspension; atelocollagen isolated and purified according to the method described in Korean Patent Publication No. 10-2011-0125772 or commercially available atelocollagen), 1.5 wt % of cationized atelocollagen solution (prepared in Example 1-1 and also used in the Examples described below), and 1.5 wt % of anionized atelocollagen solution (prepared according to Example 1-2 and also used in the Examples described below) were prepared and adjusted to pH 7.4.

2) Each of the atelocollagen suspension, the cationized atelocollagen solution and the anionized atelocollagen solution prepared in step 1) was applied to a multi-well culture dish and completely dried.

3) About 50 rat pancreatic islet cells were seeded onto each of the cationized atelocollagen scaffold, the atelocollagen scaffold and the anionized atelocollagen scaffold formed on the culture dishes in step 2), and were then cultured in a CO₂ incubator at 37° C. by adding 1 ml of RPMI-1640 medium containing 10% FBS and 1% antibiotics. In addition, pancreatic islet cells were also seeded onto a poly-L-lysine-treated culture dish and an untreated negative control culture dish and cultured in the same manner as described above. Also, the culture of the pancreatic islet cells on the culture dishes was observed.

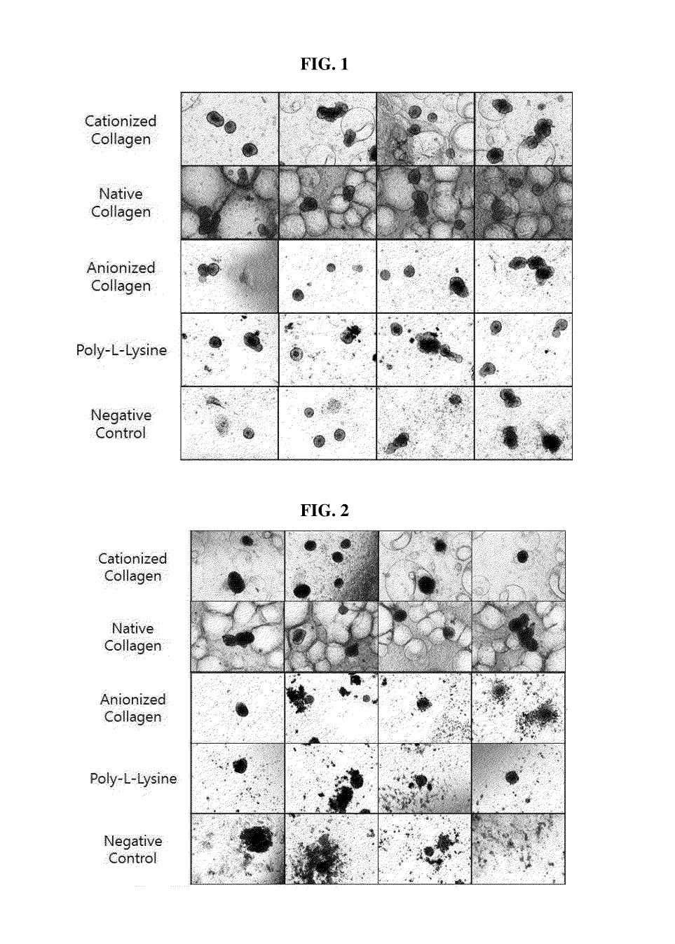

FIG. 1 is a microscopic image obtained using a CKX41 Olympus microscope (Olympus, Tokyo, Japan) at 1 week after culture of the pancreatic islet cells on the culture dishes according to the above-described process. As can be seen in FIG. 1, the pancreatic islet cells cultured on the negative control culture dish, the poly-L-lysine-treated culture dish and the anionized atelocollagen scaffold formed on the culture dish started to burst and die.

FIG. 2 is a microscopic image obtained using a CKX41 Olympus microscope (Olympus, Tokyo, Japan) at 5 weeks after culture of the pancreatic islet cells on the culture dishes. As can be seen in FIG. 2, the pancreatic islet cells, cultured on the culture dish having the anionized collagen scaffold formed thereon and the culture dish treated with poly-L-lysine, were mostly dead, similar to the negative control, but the pancreatic islet cells, cultured on the culture dish having the cationized collagen scaffold formed thereon and the culture dish having the native collagen scaffold formed thereon, mostly maintained their morphology.

Thus, it can be seen that, in contrast with general cells that are easily cultured on an ionized atelocollagen scaffold, pancreatic islet cells are not easily cultured and are mostly dead on a scaffold made of anionized atelocollagen, but show high viability while maintaining their morphology on a scaffold made of cationized atelocollagen.

### Example 3

### Induction of Insulin Secretion from Pancreatic Islet Cells by Glucose Stimulation

In order to demonstrate the superiority of the method of culturing pancreatic islet cells using cationized atelocollagen according to the present invention, pancreatic islet cells were cultured on a collagen scaffold in the following manner, and insulin secretion from the pancreatic islet cells cultured on the collagen scaffold was induced.

1) Pancreatic islet cells (divided into five groups in total) were cultured according to the procedure of Example 2 for one day, and then the medium was removed. Next, the cells were washed with KRHB (Kreb's and Ringer's HEPES Bicarbonate, pH 7.4) buffer, and the KRHB buffer was removed.

2) 1 ml of KRHB buffer was added to the pancreatic islet cells which were then cultured in a CO₂ incubator at 37° C. for 30 minutes, and then the KRHB buffer was removed and 1 ml of KRHB buffer containing 3.3 mM glucose was added to the cells. Next, the pancreatic islet cells were cultured in a CO₂ incubator at 37° C. for 1 hour, and then the glucose-containing KRHB buffer was taken and freeze-stored.

3) Also, 1 ml of KRHB buffer containing 20 mM glucose was added to pancreatic islet cells which were then cultured in a CO₂ incubator at 37° C. for 1 hour. Next, the glucose-containing KRHB buffer was taken and freeze-stored.

4) 1 m of RPMI-1640 medium was added to pancreatic islet cells, which were then cultured in a CO₂ incubator at 37° C. for 6 days and subjected to glucose stimulation as described in steps 2) and 3). Next, the cells were subjected to glucose stimulation at 1-week intervals for 8 weeks.

### Example 4

### Measurement of Glucose Stimulation Index (GSI)

Pancreatic islet cells (divided into five groups in total) cultured according to the procedure of Example 2 were stimulated with glucose according to the procedure of Example 3, and then the glucose-dependent insulin secretory activity of the cells was measured.

After performing glucose stimulation according to steps 2) and 3) of Example 3, the taken buffer solutions were diluted at 1/100 and subjected to ELISA (enzyme-linked immunosorbent assay).

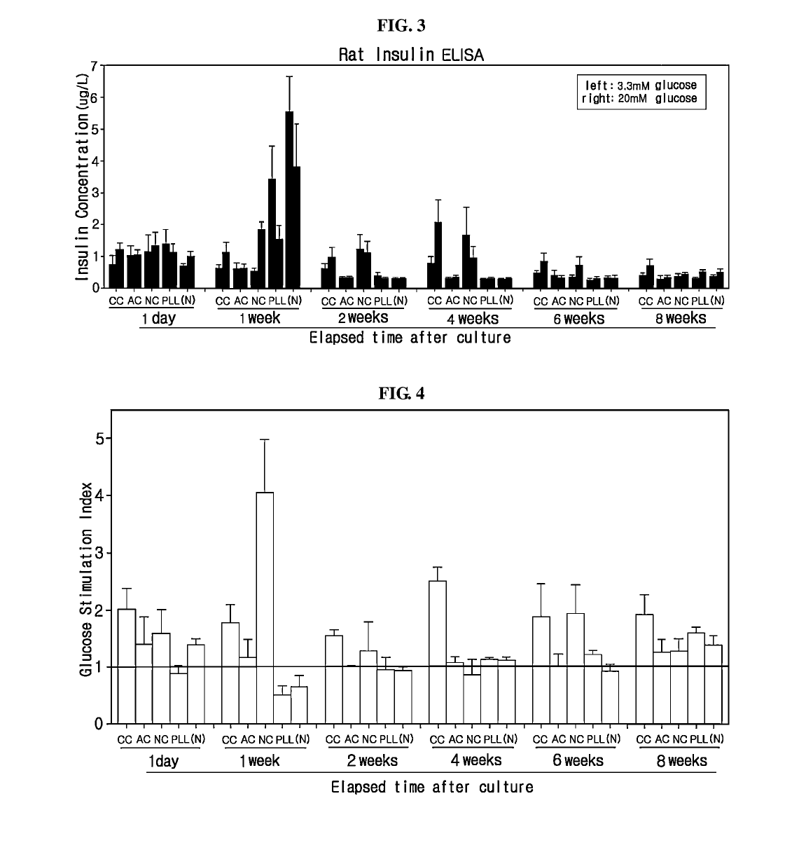

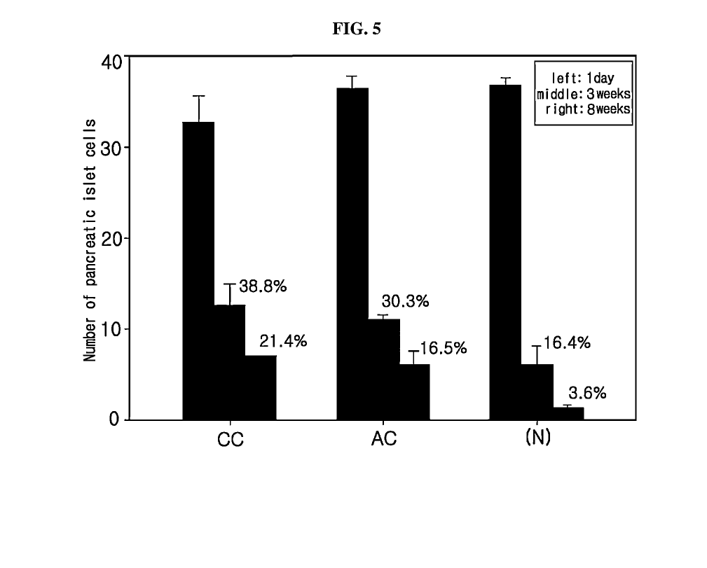

FIG. 3 shows a comparison of insulin secretion in terms of insulin concentration measured after low concentration (3.3 mM) glucose stimulation and high concentration (20 mM) glucose stimulation of pancreatic islet cells cultured on each of cationized atelocollagen(CC), anionized atelocollagen(AC), native atelocollagen(NC), poly-L-lysine(PLL) and negative control(N). FIG. 4 shows a comparison of insulin secretion in terms of glucose stimulation index measured after glucose stimulation of pancreatic islet cells cultured on each of cationized atelocollagen(CC), anionized atelocollagen(AC), native atelocollagen(NC), poly-L-lysine(PLL) and negative control(N).

As can be seen from the results in FIGS. 3 and 4, insulin secretion from the pancreatic islet cells at 1 day after culture was similar between the pancreatic islet cells, and insulin secretion from the pancreatic islet cells at 1 week after culture was the highest in the pancreatic islet cells cultured in the negative control(N) culture dish and was higher in the order of the pancreatic islet cells cultured in the culture dishes treated with poly-L-lysine(PLL), native atelocollagen(NC), cationized atelocollagen(CC) and anionized atelocollagen(AC). However, insulin secretion from the pancreatic islet cells cultured on the negative control and poly-L-lysine was glucose-independent.

Also, insulin secretion from the pancreatic islet cells at 2 weeks after culture was the highest in the pancreatic islet cells cultured in the culture dish treated with the native atelocollagen(NC) and was higher in the order of the pancreatic islet cells cultured in the cationized atelocollagen(CC)-treated culture dish and the pancreatic islet cells cultured in the poly-L-lysine-treated culture dish. However, insulin secretion from the pancreatic islet cells cultured in the native atelocollagen(NC)-treated culture dish was glucose-independent.

In addition, insulin secretion from the pancreatic islet cells at 4 weeks after culture was the highest in the pancreatic islet cells cultured in the cationized atelocollagen(CC)-treated culture dish and was second higher in the pancreatic islet cells cultured in the native atelocollagen(NC)-treated culture dish. However, insulin secretion from the pancreatic islet cells cultured in the native atelocollagen(NC)-treated culture dish was glucose-independent.

Taken together, such results indicate that only the pancreatic islet cell group cultured in the cationized atelocollagen(CC)-treated culture dish showed a certain level of glucose-dependent insulin secretion throughout the culture period. Accordingly, it was confirmed that the glucose-dependent insulin secretory activity of the pancreatic islet cells cultured on the cationized atelocollagen scaffold prepared by cationizing atelocollagen is much higher than the glucose-dependent insulin secretory activity of the pancreatic islet cells cultured on the scaffold made of native atelocollagen or anionized atelocollagen.

### Example 5

### Culture of Pancreatic Islet Cells on Crosslinked Collagen Scaffold

In order to confirm the superiority of the method of culturing pancreatic islet cells using crosslinked atelocollagen according to the present invention, pancreatic islet cells were cultured on a crosslinked collagen scaffold in the following manner.

1) 1.5 wt % of type I atelocollagen suspension (i.e. native atelocollagen suspension), 1.5 wt % of cationized atelocollagen solution and 1.5 wt % of anionized atelocollagen solution were prepared and adjusted to pH 7.4.

2) Each of the atelocollagen suspension, the cationized atelocollagen solution and the anionized atelocollagen solution prepared in step 1) was applied to a multi-well culture dish and completely dried.

3) 1 ml of 200 mM EDC solution in 95% ethanol was added to each of the atelocollagen scaffolds formed on each of the multi-well culture dishes in step 2), and then allowed to react at 4° C. for 24 hours to induce crosslinking of the atelocollagen scaffolds.

4) After completion of step 3), the multi-well culture dishes were washed 10 times with 1×PBS to remove ethanol and EDC.

5) About 50 rat pancreatic islet cells were seeded onto each of the crosslinked cationized atelocollagen scaffold, the crosslinked atelocollagen scaffold(native) and the crosslinked anionized atelocollagen scaffold formed in step 3), and were then cultured in a CO₂ incubator at 37° C. by adding 1 ml of RPMI-1640 medium containing 10% FBS and 1% antibiotics. For comparison, pancreatic islet cells were seeded and cultured in each of a cationized atelocollagen-coated culture dish, a native atelocollagen-coated culture dish and a negative control culture dish in the same manner as above. However, pancreatic islet cells cultured on a culture dish coated with non-crosslinked anionized atelocollagen prepared using succinic anhydride were excluded from the experiment because the anionized atelocollagen coating was dissolved out by the culture medium.

### Example 6

### Induction of Insulin Secretion from Pancreatic Islet Cells by Glucose Stimulation

In order to confirm the superiority of the method of culturing pancreatic islet cells using crosslinked atelocollagen according to the present invention, pancreatic islet cells were cultured on a collagen scaffold in the following manner, and insulin secretion from the pancreatic islet cells cultured on the collagen scaffold was induced.

1) Pancreatic islet cells (divided into six groups in total) were cultured according to the procedure of Example 5 for one day, and then the medium was removed. Next, the cells were washed with KRHB (Kreb's and Ringer's HEPES Bicarbonate, pH 7.4) buffer, and the KRHB buffer was removed.

2) 1 ml of KRHB buffer was added to the pancreatic islet cells which were then cultured in a CO₂ incubator at 37° C. for 30 minutes, and then the KRHB buffer was removed and 1 ml of KRHB buffer containing 3.3 mM glucose was added to the cells. Next, the pancreatic islet cells were cultured in a CO₂ incubator at 37° C. for 1 hour, and then the glucose-containing KRHB buffer was taken and freeze-stored.

3) Also, 1 ml of KRHB buffer containing 20 mM glucose was added to pancreatic islet cells which were then cultured in a CO₂ incubator at 37° C. for 1 hour. Next, the glucose-containing KRHB buffer was taken and freeze-stored.

4) 1 m of RPMI-1640 medium was added to pancreatic islet cells, which were then cultured in a CO₂ incubator at 37° C. for 6 days and subjected to glucose stimulation as described in steps 2) and 3). Next, the cells were subjected to glucose stimulation at 1-week intervals for 4 weeks.

### Example 7

### Measurement of Glucose Stimulation Index (GSI)

Pancreatic islet cells (divided into six groups in total) cultured according to the procedure of Example 5 were stimulated with glucose according to the procedure of Example 6, and then the glucose-dependent insulin secretory activity of the cells was measured.

After performing glucose stimulation according to steps 2) and 3) of Example 6, the taken buffer solutions were diluted at 1/100 and subjected to ELISA (enzyme-linked immunosorbent assay).

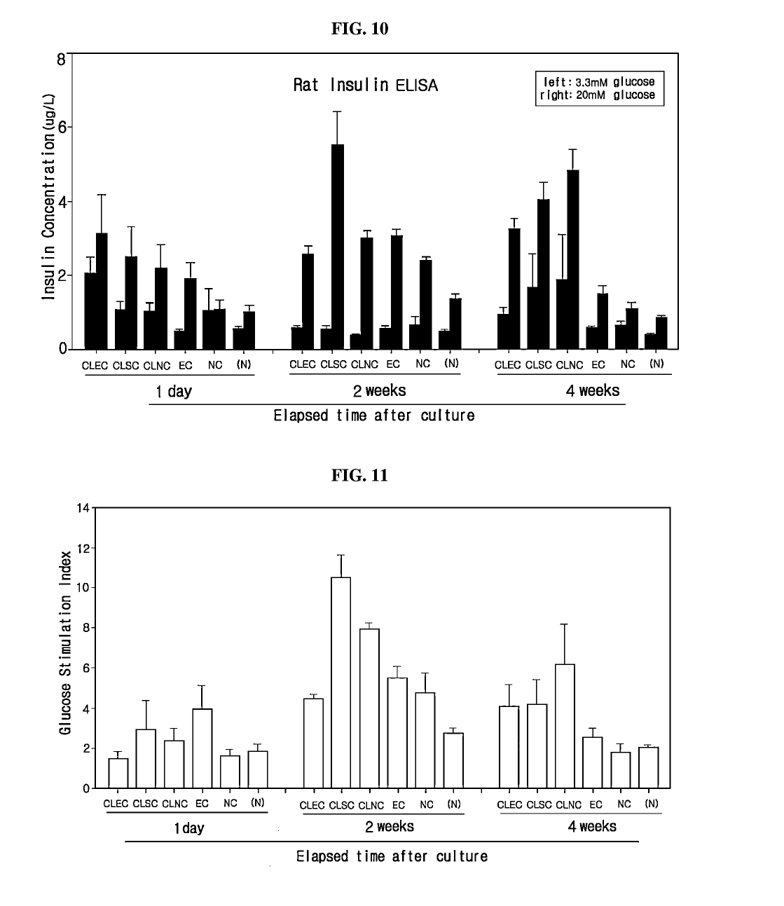

FIG. 10 shows a comparison of insulin secretion in terms of insulin concentration measured after low concentration (3.3 mM) and high concentration (20 mM) glucose stimulation of pancreatic islet cells cultured on each of crosslinked cationized atelocollagen(CLEC), crosslinked anionized atelocollagen(CLSC), crosslinked native atelocollagen(CLNC), cationized atelocollagen(EC), native atelocollagen(NC) and negative control(N). FIG. 11 shows a comparison of insulin secretion in terms of glucose stimulation index measured after glucose stimulation of pancreatic islet cells cultured on each of crosslinked cationized atelocollagen(CLEC), crosslinked anionized atelocollagen(CLSC), crosslinked native atelocollagen(CLNC), cationized atelocollagen(EC), native atelocollagen(NC) and negative control(N).

As can be seen from the results in FIGS. 10 and 11, insulin secretion from the pancreatic islet cells cultured in the culture dishes having the crosslinked cationized atelocollagen scaffold(CLEC), the crosslinked anionized atelocollagen scaffold(CLSC) and the crosslinked atelocollagen scaffold(native)(CLNC) formed thereon, respectively, was generally higher than insulin secretion from pancreatic islet cells cultured in a culture dish coated with non-crosslinked cationized atelocollagen(EC) or native atelocollagen(NC). Also, this tendency was more evident at 4 weeks after culture of the pancreatic islet cells stimulated with high concentration glucose.

Taken together, such results indicate that the pancreatic islet cell group cultured in the culture dish having the crosslinked atelocollagen scaffold formed thereon showed a high level of glucose-dependent insulin secretion throughout the culture period. Accordingly, it was confirmed that the glucose-dependent insulin secretory activity of pancreatic islet cells cultured on the crosslinked atelocollagen scaffold is higher than the glucose-dependent insulin secretory activity of pancreatic islet cells cultured on the non-crosslinked atelocollagen.

### Example 8

### Measurement and Comparison of Viability of Pancreatic Islet Cells

The results in FIGS. 1 and 2 indicate that pancreatic islet cells are not easily cultured and are mostly dead on a scaffold made of anionized atelocollagen, but show high viability while maintaining their morphology on a scaffold made of cationized atelocollagen. In this Example, quantification of the viability of pancreatic islet cells was performed.

Specifically, in this Example, in order to measure the viability of pancreatic islet cells cultured on each of a cationized atelocollagen scaffold(CC), an anionized atelocollagen scaffold(AC) and a negative control(N), the cell number of pancreatic islet cells in each of the culture groups was measured at 1 day, 3 weeks and 8 weeks after culture and compared between the culture groups. The results of the measurement are shown by graphs in FIG. 5. As can be seen in FIG. 5, the pancreatic islet cell group cultured on the cationized atelocollagen scaffold(CC) showed a viability of 38.8% at 3 weeks after culture, whereas the pancreatic islet cell group cultured on the anionized atelocollagen scaffold(AC) showed a viability of 30.3%, and the negative control group showed a viability of 16.4%. Accordingly, it was confirmed that the pancreatic islet cell group cultured on the cationized atelocollagen scaffold(CC) shows high viability.

In addition, the pancreatic islet cell group cultured on the cationized atelocollagen scaffold(CC) showed a viability of 21.4% at 8 weeks after culture, whereas the pancreatic islet cell group cultured on the anionized atelocollagen scaffold(AC) showed a viability of 16.5%, and the negative control group showed a viability of 3.6%. Accordingly, it was confirmed that the pancreatic islet cell group cultured on the cationized atelocollagen scaffold(CC) shows high viability as compared with other pancreatic islet cell groups and can be maintained at high viability even when these cells are cultured for a long period of time. Thus, it can be confirmed that the method of culturing pancreatic islet cells using cationized atelocollagen and the carrier for pancreatic islet cell transplantation according to the present invention as described below can resolve the problem of insufficient supply of pancreatic islet cells for treatment of diabetes.

### Example 9

### Examination of Effect of Ionized Collagen on Proliferation of Cells

In this Example, in order to examine whether the effect of cationized atelocollagen on increases in the viability and glucose-dependent insulin secretion of pancreatic islet cells cultured on a cationized atelocollagen scaffold appears in all types of cells or whether it appears in a specific type of cell, the following experiment was performed.

(1) Coating with Ionized Collagen

1) 1.5 wt % of type I atelocollagen suspension, 1.5 wt % of cationized atelocollagen solution and 1.5 wt % of anionized atelocollagen solution were prepared and adjusted to pH 7.4.

2) Each of the atelocollagen suspension, the cationized atelocollagen solution and the anionized atelocollagen solution prepared in step 1) was applied to a multi-well culture dish and completely dried.

3) 200 mM EDC solution in 95% ethanol was dispensed into each well of the multi-well culture dishes prepared in step 2), and then each well was incubated for 24 hours to induce crosslinking of the collagen.

4) Each well treated in step 3) was washed 10 times with 1×PBS buffer to remove EDC and ethanol.

5) After completion of step 4), the multi-well culture dishes were sterilized with UV light for 1 hour.

(2) Cell Culture and MTT Assay

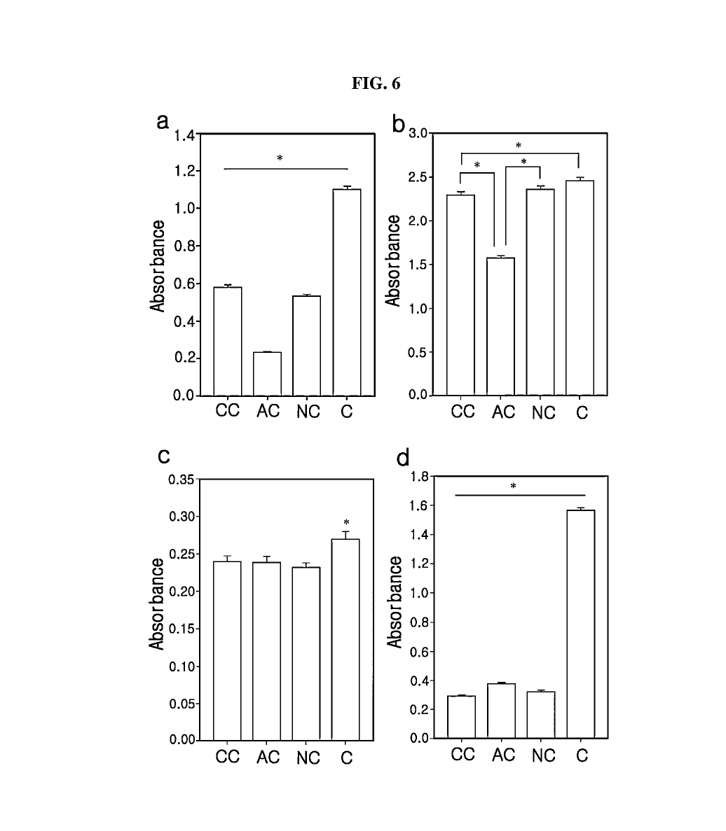

1) Each of culture dishes prepared in the above process (1) and tissue culture medium-treated culture dishes (indicated by “C” in FIG. 6) was seeded with mouse fibroblast L929 cells (0.8×10⁴ cells) and rat MSC cells (0.8×10⁴ cells), which were then cultured for 3 days and 7 days.

2) A solution of MTT reagent (thiazolyl blue tetrazolium bromide, 5 mg/ml) in 1×PBS buffer was added to each cell culture at a ratio of 1/10, and then the cells were cultured at 37° C. for 4 hours.

3) After removing the medium, 1 ml of DMSO was added to dissolve the reaction product, and at 3 days and 7 days after culture, an MTT assay for the L929 cells and the rat MSC cells was performed by measuring absorbance at 540 nm.

The results of the measurement are shown in FIG. 6 (n=4, mean±SE, * p<0.05). FIG. 6a shows the MTT assay results obtained by measuring absorbance at 3 days after culture of L929 cells, and FIG. 6b shows the MTT assay results obtained by measuring absorbance at 7 days after culture of L929 cells. In addition, FIG. 6c shows the MTT assay results obtained by measuring absorbance at 3 days after culture of rat MSC cells, and FIG. 6d shows the MTT assay results obtained by measuring absorbance at 7 days after culture of rat MSC cells.

As can be seen from the MTT assay results in FIG. 6, the proliferation of L929 cells on the anionized atelocollagen film(AC) decreased as the culture time increased (at 7 days after culture), but no significant difference in the proliferation of the cells on the cationized atelocollagen film(CC) and the native atelocollagen film(NC) was observed (see FIG. 6b). On the other hand, as the culture time increased (at 7 days after culture), the proliferation of the rat MSC cells cultured on the anionized atelocollagen film(AC) increased as compared with the proliferation of the rat MSC cells cultured on the cationized atelocollagen film(CC) and the native atelocollagen film(NC) (see FIG. 6d).

In other words, the results of proliferation of the L929 cells and the rat MSC cells at 7 days after culture of the cells on the atelocollagen films did completely differ between the two types of cells. Specifically, it could be seen that the proliferation of the L929 cells cultured on the anionized atelocollagen film(AC) was reduced as compared with the proliferation of the L929 cells cultured on other atelocollagen films(CC and NC), whereas the proliferation of the rat MSC cells cultured on the anionized atelocollagen film(AC) increased as compared with the proliferation of the rat MSC cells cultured on other atelocollagen films(CC and NC). From such results, it can be seen that cell proliferation is cell-specific and is not associated directly with cell viability.

Accordingly, it cannot be concluded that, even though cell adhesion and proliferation increase during cell culture, these increases are associated with an increase in cell viability and a positive effect on the function of the cells. For this reason, in the technical field to which the present invention pertains, the development of a novel method for culturing pancreatic islet cells and a highly stable carrier for pancreatic islet cell transplantation is required from the viewpoint of increasing the viability and glucose-dependent insulation secretion of pancreatic islet cells, but not the viewpoint of cell proliferation that is cell-specific.

### Example 10

### Method of Preparing a Carrier for Pancreatic Islet Cell Transplantation Using Cationized Atelocollagen

In this Example, a highly stable carrier for pancreatic islet cell transplantation, which comprises cationized atelocollagen and alginate, was prepared in the following manner.

1) A cationized atelocollagen solution and an alginate solution were mixed with each other to prepare a mixed solution having a cationized atelocollagen concentration of 1% (w/v) and an alginate concentration of 2% (w/v), and pancreatic islet cells were added to the mixed solution. In addition, a cationized atelocollagen solution and an alginate solution were mixed with each other to prepare a mixed solution having a cationized atelocollagen concentration of 0.5% (w/v) and an alginate concentration of 2% (w/v), and pancreatic islet cells were added to the mixed solution. Further, pancreatic islet cells were added to 2% alginate solution prepared as a control.

2) The pancreatic islet cell complex comprising pancreatic islet cells mixed with and surrounded by the mixed solution of cationized atelocollagen and alginate was formed into a small drop, which was then immersed in 100 mM CaCl₂ solution containing 10 mM HEPES (4-(2-hydroxyethyl)-1-piperazine ethane sulfonic acid) and 2 mM potassium chloride for 5 minutes, thereby producing a cationized atelocollagen/alginate bead. Meanwhile, the cell complex comprising pancreatic islet cells mixed with 2% alginate solution as a control was treated in the same manner as above, thereby producing an alginate bead.

3) The beads produced in step 2) were washed with KRH buffer (Krebs-Ringer-HEPES-glucose-glutamine buffer) for 1 minute, after which the beads were immersed in 0.1% poly-L-lysine solution for 10 minutes, and then washed three times with Ca²⁺-free KRH buffer for 3 minutes each time.

4) The beads treated in step 3) were immersed in 0.2% alginate solution for 5 minutes, and then allowed to stand in Ca²⁺-free KRH buffer containing 1 mM EGTA for 10 minutes to liquefy the alginate. Next, the beads were washed three times with KRH buffer, thereby producing a carrier for pancreatic islet cell transplantation according to an embodiment of the present invention and a control carrier.

### Example 11

### Examination of Increases in Glucose-Dependent Insulin Secretion and Pancreatic Islet Cell Viability in the Carrier for Pancreatic Islet Cell Transplantation According to the Present Invention

In order to examine increases in glucose-dependent insulin secretion and pancreatic islet cell viability in the carrier for pancreatic islet cell transplantation according to the present invention, each of the carrier for pancreatic islet cell transplantation comprising cationized atelocollagen/alginate and the alginate bead that is a control carrier, prepared in Example 10, was incubated in RPMI 1640 medium containing 10% FBS (fetal bovine serum) and 1% antibiotics.

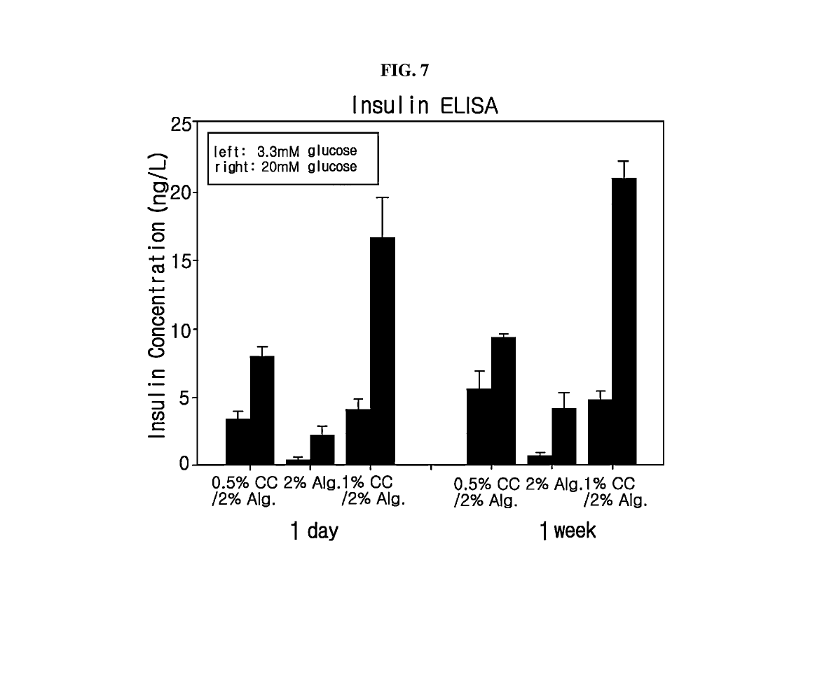

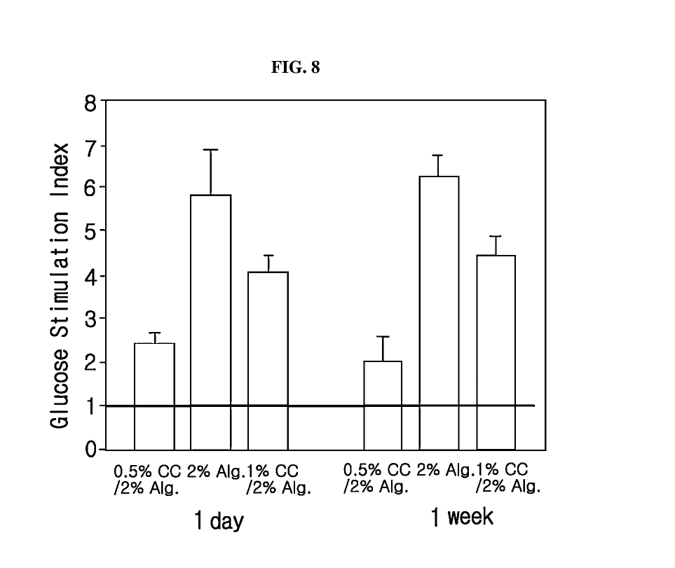

Specifically, in order to examine an increase in glucose-dependent insulin secretion from the carrier for pancreatic islet cell transplantation according to the present invention, a glucose stimulation test as described in Example 3 was conducted, and insulin secretion and glucose-dependent insulin secretion from the carriers were measured. The results of the measurement are shown in FIGS. 7 and 8.

Specifically, the pancreatic islet cells in each of the carrier for pancreatic islet cell transplantation comprising cationized atelocollagen/alginate according to the present invention and the alginate bead that is a control carrier were cultured. At 1 day and 1 week after culture, insulin secretion after glucose stimulation was measured as described in Example 4. FIG. 7 shows the results of measuring insulin concentration, and FIG. 8 shows the results of measuring glucose stimulation index. As can be seen from these results, insulin secretion from the pancreatic islet cells contained in the carrier for pancreatic islet cell transplantation comprising cationized atelocollagen/alginate according to the present invention was generally increased as compared with insulin secretion from the alginate bead that is a control carrier. Also, it could be observed that, as the content of cationized atelocollagen increased, insulin secretion induced by high concentration glucose stimulation increased.

Meanwhile, in order to confirm the increased viability of pancreatic islet cells contained in the carrier for pancreatic islet cells according to the present invention, FDA/PI staining was performed. FDA/PI staining is a staining method well known in the art, which is performed in order to microscopically observe dead cells and viable cells. In this Example, a solution of 0.05 mg/ml of FDA (fluorescein diacetate) in acetone and a solution of 0.05 mg/ml of PI (propidium iodide) in PBS were used. 20 μL of the PI solution was added to the cell culture and sufficiently shaken for 30 seconds, and then 20 μL of the FDA solution was added thereto and sufficiently shaken for 30 seconds. Thereafter, the cells were washed twice with PBS and observed with a fluorescence microscope (Leica, CM1850). Herein, viable pancreatic islet cells emit green fluorescence by FDA/PI staining, and dead pancreatic islet cells emit red fluorescence by FDA/PI staining.

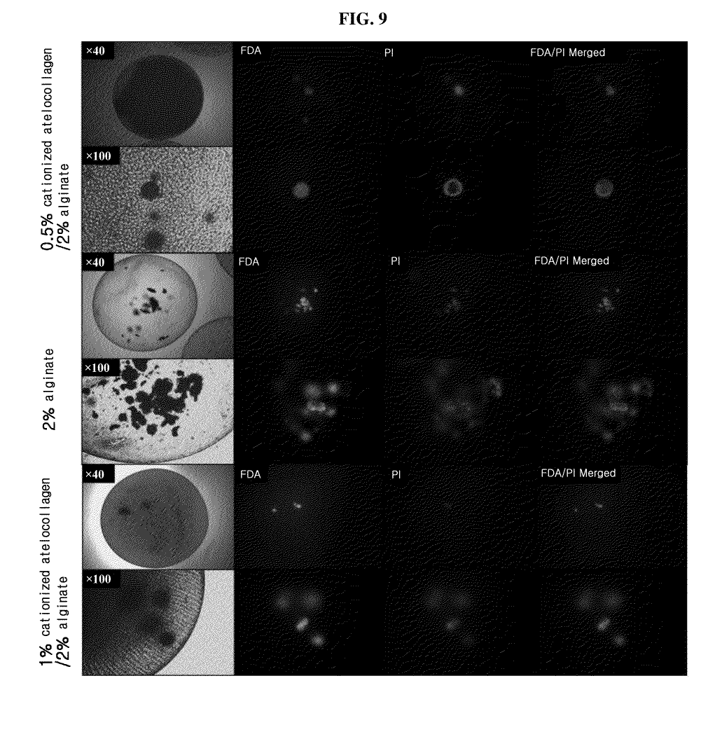

FIG. 9 shows fluorescence microscope images obtained after FDA/PI staining of the pancreatic islet cells contained in the cationized atelocollagen/alginate bead, which is the carrier for pancreatic islet cell transplantation according to the present invention, and the pancreatic islet cells contained in the alginate bead that is a control carrier.

As can be seen from the images of FIG. 9, the pancreatic islet cells contained in the cationized atelocollagen/alginate bead that is the carrier for pancreatic islet cell transplantation according to the present invention have much green color and less red color as compared with the pancreatic islet cells contained in the alginate bead that is a control carrier. Accordingly, it was confirmed that the ratio of viable cells in the pancreatic islet cells contained in the cationized atelocollagen/alginate bead is higher than that in the alginate bead.

Taken together, such results indicate that the carrier for pancreatic islet cell transplantation that comprises cationized atelocollagen and alginate according to the present invention has advantages that it is highly stable and can increase the viability of cultured and transplanted pancreatic islet cells while increasing glucose-dependent insulin secretion.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

We claim:

1. A method for preparing a carrier for pancreatic islet cell transplantation, the method comprising:
(a) mixing a cationized atelocollagen solution with an alginate solution to prepare a mixed solution;
(b) adding pancreatic islet cells to the mixed solution of step (a);
(c) allowing the pancreatic islet cells to be mixed with and surrounded by the mixed solution of step (a) to form a pancreatic islet cell complex comprising the pancreatic islet cells surrounded by the mixed solution of step (a); and
(d) immersing the pancreatic islet cell complex obtained by step (c) comprising the pancreatic islet cells surrounded by the mixed solution of step (a), in a chelating agent solution to chelate the cationized atelocollagen and the alginate in the mixed solution and to produce a cationized atelocollagen/alginate bead containing the pancreatic islet cells therein.

2. The method of claim 1, further comprising forming an immune barrier on the cationized atelocollagen/alginate bead produced in step (d).

3. The method of claim 2, wherein the immune barrier is formed by immersing the atelocollagen/alginate bead of step (d) in a poly-L-lysine solution.

4. The method of claim 2, further comprising a step of forming an additional alginate coating on the immune barrier.

5. The method of any one of claims 1 to 4, wherein the chelating agent is a metal ion chelating agent that chelates the cationized atelocollagen and the alginate in the mixed solution of the cationized atelocollagen solution and the alginate solution.

6. The method of any one of claims 1 to 4, wherein the concentration ratio between the cationized atelocollagen solution and the alginate solution, which are mixed with each other in step (a), is 1:2.

7. A carrier for pancreatic islet cell transplantation prepared according to the method of claim 1.

8. The carrier of claim 7, further comprising an immune barrier formed on the cationized atelocollagen/alginate bead.

9. The carrier of claim 8, further comprising an alginate coating formed on the immune barrier.

10. An artificial pancreas comprising:
a carrier for pancreatic islet cell transplantation, which is prepared according to the method of claim 1 and is in the form of the cationized atelocollagen/alginate bead; and
pancreatic islet cells contained in the carrier for pancreatic islet cell transplantation.

11. The artificial pancreas of claim 10, further comprising an immune barrier formed on the cationized atelocollagen/alginate bead.

12. The artificial pancreas of claim 11, further comprising an alginate coating formed on the immune barrier.

13. A method for culturing pancreatic islet cells using atelocollagen, the method comprising:
(a) preparing a cationized atelocollagen solution;
(b) either seeding pancreatic islet cells into the cationized atelocollagen solution, or applying the cationized atelocollagen solution to a culture vessel, drying the applied cationized atelocollagen solution to form a cationized atelocollagen scaffold, and seeding pancreatic islet cells onto the cationized atelocollagen scaffold; and
(c) culturing the seeded pancreatic islet cells of step (b) in the cationized atelocollagen solution or on the cationized atelocollagen scaffold.

14. A method for culturing pancreatic islet cells using atelocollagen, the method comprising:
(a) preparing an atelocollagen solution;
(b) applying the atelocollagen solution to a culture vessel, drying the applied atelocollagen solution to form an atelocollagen scaffold, crosslinking the atelocollagen scaffold, and seeding pancreatic islet cells onto the crosslinked atelocollagen scaffold; and
(c) culturing the seeded pancreatic islet cells of step (b) on the crosslinked atelocollagen scaffold.

15. The method of claim 14, wherein the crosslinking of the atelocollagen scaffold in step (b) is induced by reacting the atelocollagen scaffold with a solution containing a crosslinking agent.

16. The method of claim 15, wherein the crosslinking agent is 1-ethyl-3-(3-dimethyl aminopropyl)carbodiimide (EDC) or glutaraldehyde.
